(19) United States
(12) Patent Application Publication
(10) Pub. No.: US 2007/0078375 A1
(43) Pub. Date: Apr. 5, 2007

(54) Iontophoretic delivery of active agents conjugated to nanoparticles

(75) Inventor: Gregory A. Smith, Sammamish, WA (US)

Correspondence Address: SEED INTELLECTUAL PROPERTY LAW GROUP PLLC, 701 FIFTH AVE, SUITE 5400, SEATTLE, WA, 98104, US

(73) Assignee: Transcutaneous Technologies Inc., Tokyo (JP)

(21) Appl. No.: 11/540,950

(22) Filed: Sep. 28, 2006

Related U.S. Application Data
(60) Provisional application No. 60/722,260, filed on Sep. 30, 2005.

(51) Int. Cl. A61N 1/30

(52) U.S. Cl. 604/20

(57) ABSTRACT

An iontophoresis device is provided to delivery active agents to a biological interface, the iontophoresis device comprising: an active electrode element operable to provide an electrical potential; and an inner active agent reservoir comprising a plurality of nanoparticles, each nanoparticles being conjugated to a plurality of active agents via respective linkers.

### CROSS-REFERENCE TO RELATED APPLICATION

This application claims the benefit under 35 U.S.C. § 119(e) of U.S. Provisional Patent Application No. 60/722,260, filed Sep. 30, 2005.

### BACKGROUND OF THE INVENTION

1. Field of the Invention

This disclosure generally relates to the field of iontophoresis, and more particularly to the delivery of active agents such as therapeutic agents under the influence of electromotive force.

2. Description of the Related Art

Iontophoresis employs an electromotive force to transfer an active agent such as an ionic drug or other therapeutic agent to a biological interface, for example skin or mucus membrane.

Iontophoresis devices typically include an active electrode assembly and a counter electrode assembly, each coupled to opposite poles or terminals of a power source, for example a chemical battery. Each electrode assembly typically includes a respective electrode element to apply an electromotive force. Such electrode elements often comprise a sacrificial element or compound, for example silver or silver chloride.

The active agent may be either cation or anion, and the power source can be configured to apply the appropriate voltage polarity based on the polarity of the active agent. Iontophoresis may be advantageously used to enhance or control the delivery rate of the active agent. As discussed in U.S. Pat. No. 5,395,310, the active agent may be stored in a reservoir such as a cavity. Alternatively, the active agent may be stored in a reservoir such as a porous structure or a gel. Also as discussed in U.S. Pat. No. 5,395,310, an ion exchange membrane may be positioned to serve as a polarity selective barrier between the active agent reservoir and the biological interface.

The commercial acceptance of iontophoresis devices is further dependent on a variety of factors, such as cost to manufacture, shelf life or stability during storage, shelf life or stability during storage, efficiency and/or timeliness of active agent delivery and release, biological capability and/or disposal issues. Of these, the timeliness of delivery and release pattern of the active agents in the biological tissue or circulation is of particular interest, because these characteristics are directly associated with the pharmacokinetics and therapeutic efficacy of the active agents. An iontophoresis device that addresses one or more of the above factors is desirable.

### BRIEF SUMMARY OF THE INVENTION

In one embodiment, an iontophoresis device is provided to delivery active agents to a biological interface, the iontophoresis device comprising: an active electrode element operable to provide an electrical potential; and an inner active agent reservoir comprising a plurality of nanoparticles, each nanoparticles being conjugated to a plurality of active agents via respective linkers. The nanoparticle conjugated preferably form an active agent depot in the biological tissue underlying the biological interface. Enzymatic cleavage can cause the active agents to be detached from the nanoparticle in a sustained fashion.

A further embodiment describes a method for transdermal administration of an active agent by iontophoresis, the method comprising: positioning an active electrode assembly and a counter electrode assembly of an iontophoresis device on a biological interface of a subject, the active electrode assembly further including an active electrode element operable to provide an electrical potential; and an inner active agent reservoir comprising a plurality of nanoparticles, each nanoparticles being conjugated to one or more active agents via respective linkers; and applying a sufficient amount of current to administer a therapeutically effective amount of the active agents conjugated to the nanoparticles in the subject for a limited period of time.

### BRIEF DESCRIPTION OF THE SEVERAL VIEWS OF THE DRAWINGS

In the drawings, identical reference numbers identify similar elements or acts. The sizes and relative positions of elements in the drawings are not necessarily drawn to scale. For example, the shapes of various elements and angles are not drawn to scale, and some of these elements are arbitrarily enlarged and positioned to improve drawing legibility. Further, the particular shapes of the elements as drawn, are not intended to convey any information regarding the actual shape of the particular elements, and have been solely selected for ease of recognition in the drawings.

FIG. 1 is a block diagram of an iontophoresis device comprising active and counter electrode assemblies according to one illustrated embodiment.

FIG. 2 is a block diagram of the iontophoresis device of FIG. 1 positioned on a biological interface, with the outer release liner removed to expose the active agent according to one illustrated embodiment.

### DETAILED DESCRIPTION OF THE INVENTION

The iontophoresis device described herein comprises active agents covalently conjugated to nanoparticles housed in an active agent reservoir. In particular, an active agent can be linked or tethered to the surface of a biocompatible nanoparticle via a linker to form a conjugate. The conjugates can be transported across a biological interface, such as skin or mucus membrane, by an iontophoretic or microneedle device. Sustained release of the free active agents is possible when the active agents are cleaved from the nanoparticles by naturally occurring enzymes within the skin.

Nanoparticles (e.g., gold) conjugated to active agents such as antibodies are known to be administered intravenously. The nanoparticles can be preferentially delivered to target tissues due to their high vascular permeability. However, the nanoparticles administered in this fashion can be sequestered by macrophage or otherwise rapidly eliminated by the immune systems or organs such as liver and spleen. In contrast, subcutaneously administered nanoparticles are cleared by the body much more slowly as compared to i.v. administration. This allows for the build up of a depot wherein the active agents undergo sustained release as they are cleaved by endogenous enzymes. This depot effect is particular beneficial with regard to the sustained release of small molecular weight, hydrophilic active agents, which otherwise tend to be rapidly absorbed into the systemic circulation.

In the following description, certain specific details are set forth in order to provide a thorough understanding of various disclosed embodiments. However, one skilled in the relevant art will recognize that embodiments may be practiced without one or more of these specific details, or with other methods, components, materials, etc. In other instances, well-known structures associated with controllers including but not limited to voltage and/or current regulators have not been shown or described in detail to avoid unnecessarily obscuring descriptions of the embodiments.

Unless the context requires otherwise, throughout the specification and claims which follow, the word “comprise” and variations thereof, such as, “comprises” and “comprising” are to be construed in an open, inclusive sense, that is, as “including, but not limited to.”

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

As used herein and in the claims, the term “nanoparticles” or “nanoparticle carriers” refer to minute carrier structures that can be surface-treated to conjugate to an active agent via a linker. The nanoparticle is compatible with and sufficiently resistant to chemical and/or physical destruction by the environment of use such that a sufficient amount of the nanoparticles remains substantially intact during iontophoretic delivery. Biodegradation of the nanoparticle is permissible following the release of the active agent.

In one embodiment, nanoparticles can be solid colloidal particles ranging in sizes from 1 to 1000 nm. Nanoparticles can have any diameter less than or equal to approximately 1000 nm, including, for example, 5, 10, 15, 20, 25, 30, 50, 100, 500 and 750 nm. In another embodiment, nanoparticles can also be hollow or porous. In yet another embodiment, nanoparticles can have a core/shell structure. Other types of the nanoparticles may be employed by one skilled in the art in light of the teaching therein.

In one embodiment, the nanoparticles comprise metallic components. Typically, the metallic component is an elemental metal or a complex of metal, including semiconductive metal oxides. Examples of the nanoparticles include, but are not limited to, gold, silver or TiO₂ particles. More typically, the metallic component is gold. Unless specified otherwise, the gold nanoparticles described herein include solid or porous gold particles, hollow gold shells, and nanoparticles of core/shell structure having a gold shell deposited on a core material. The core material can be polymeric, inorganic (e.g., silica) or a different metal.

Gold nanoparticles are particularly useful as carriers of active agents in an iontophoresis device. Significantly, a gold surface can be readily chemically modified to provide a linker that can be further covalently attached to an active agent. As used herein, “covalently attached”, “couple” and “conjugate” all refer to the process of forming stable chemical bonds. In particular, gold nanoparticles can be capped with a monolayer of organic molecules possessing functional groups such as quaternary ammonium halide, amines, thiols, isothiocyanates, etc. When the organic molecules possess an additional functional group that can be further coupled to an active agent, the gold nanoparticles become a stable carrier for the active agent. Depending on the size, a gold nanoparticle may accommodate more than one active agent on its surface.

Moreover, gold nanoparticles can be surface-modified to carry net charges. For example, colloidal gold prepared in aqueous medium by chemical reduction are usually capped with anions (e.g., citrate). The negative surface charges typically provide the repulsive force between the particles, preventing them from agglomerating. Gold particles can also be modified to carry positive charges, for example, gold particles can be surface treated and stabilized by quaternary ammonium halide, (NR₄)⁺Br⁻, wherein each R can independently be an alkyl. Neutral gold nanoparticles are also within the scope contemplated herein.

Furthermore, gold particles are known for their low toxicity and may have anti-microbial property that is beneficial in its own right.

Aqueous dispersions of gold nanoparticles can be prepared. In brief, gold nanoparticles can be synthesized by reduction of gold chloride (HAuCl₄) with freshly prepared sodium citrate and allowed to boil under reflux condition. Gold nanoparticles are also commercially available from vendors such as Sigma-Aldrich (Milwaukee, Wis.).

As noted above, the nanoparticles described herein can be conjugated to active agents via a linker. The term “linker” refers to a diverse group of covalent linkages that connects the nanoparticle to an active agent. The covalent linkage can be a combination of stable chemical bonds, optionally including single, double, triple or aromatic carbon-carbon bonds, as well as carbon-nitrogen bonds, nitrogen-nitrogen bonds, carbon-oxygen bonds, carbon-sulfur bonds, phosphorus-oxygen bonds, and phosphorus-nitrogen bonds. The types of linkers suitable for the present device are not particularly limited so long as they simultaneously form stable covalent.bonds with the nanoparticles and the active agent. Additionally, the linker is liable to become detached from the active agent under certain biological conditions. For example, the bonding between a linker and an active agent can be a substrate for enzymatic degradation.

A linker is typically at least bi-functional, i.e., it comprises two functional groups, one of which is for binding to the nanoparticle, the other for binding to the active agent. The binding typically results in the formation of carboxylate ester, amide, ether, thioether, carbamate, sulfonamide, urea, or urethane moiety, in addition to thio-metal and nitrogen-metal bonds. In certain embodiments, one of the functional groups is a hydroxy and the other functional group is a thiol. A linker having a hydroxy functional group can be readily coupled to an active agent having a carboxylic acid functionality. Thiol group is known to form stable bond with gold.

In one embodiment, the linker is a polyethyleneglycol (PEG) derivative. The native form of PEG is a linear polyetherdiol that exhibits a low degree of immunogenicity and antigenicity. The polymer backbone is essentially chemically inert, and the terminal primary hydroxyl groups are available for derivatization. PEG activation and functionalization methods have been exhaustively reviewed. (e.g., Monfardini, C. and Veronese, F., Stabilization of substances in circulation. Bioconjugate Chem 9: 418-450, 1998). In particular, bi-functional PEG containing terminal hydroxy and thiol groups can be prepared according to the methods described in Biomedical Applications of Gold nanoparticles Functionalized Using Hetero-Bifunctional Poly(ethyleneglycol) Spacer, Fu, W., et. al., Mater. Res. Soc. Symp. Proc. 805, AA5.4.1, 2005, which references are incorporated herein by reference in its entirety. Surface modification of nanoparticles with PEG and its derivatives can be performed by incorporation during the production of nanoparticles, or by covalent attachment to the surface of particles. Typically, the molecularweight of the PEG segment is at least 200. More typically, the molecular weight of the PEG segment is in the range of about 500-2000 Daltons.

Generally speaking, during iontophoresis, charged or uncharged species (including the active agents and the nanoparticles) can migrate across a permeable biological interface into the underlying biological tissue. Typically, an iontophoresis device generates both electro-repulsive and electro-osmotic forces. For charged species, the migration is primarily driven by electro-repulsion between the oppositely charged active electrode and the charged species. In addition to the electro-repulsive forces, the electro-osmotic flow of a liquid (e.g., a solvent or diluent) may also contribute to transporting the charged species. In certain embodiments, the electro-osmotic solvent flow is a secondary force that can enhance the migration of the charged species. For uncharged or neutral species, the migration is primarily driven by the electro-osmotic flow of a solvent. “Active agent” refers to a compound, molecule, or treatment that elicits a biological response from any host, animal, vertebrate, or invertebrate, including for example fish, mammals, amphibians, reptiles, birds, and humans. Examples of active agents include therapeutic agents, pharmaceutical agents, pharmaceuticals (e.g., a drug, a therapeutic compound, pharmaceutical salts, and the like) non-pharmaceuticals (e.g., cosmetic substance, and the like), a vaccine, an immunological agent, a local or general anesthetic or painkiller, an antigen or a protein or peptide such as insulin, a chemotherapy agent, an anti-tumor agent.

In some embodiments, the term “active agent” further refers to the active agent, as well as its pharmacologically active salts, pharmaceutically acceptable salts, prodrugs, metabolites, analogs, and the like. In some further embodiment, the active agent includes at least one ionic, cationic, anionic, ionizable, and/or neutral therapeutic drug and/or pharmaceutical acceptable salts thereof.

In some embodiments, the active agent may include one or more “cationic active agents” that are positively charged, and/or are capable of forming positive charges in aqueous media. For example, many biologically active agents have functional groups that are readily convertible to a positive ion or can dissociate into a positively charged ion and a counter ion in an aqueous medium. Other active agents may be polarized or polarizable, that is, exhibiting a polarity at one portion relative to another portion. For instance, an active agent having an amine group can typically take the form a quaternary ammonium cation (−NR₃H⁺) at an appropriate pH, also referred to as a protonated amine. As will be discussed in detail below, many active agents, including most of the “caine” class analgesics and anesthetics, comprise amine groups. These amine groups can be present in the iontophoresis device in protonated forms.

In other embodiments, the active agents may include functional groups that can readily converted to contain negatively charges or can dissociate into a negatively charged ion and a counter ion in an aqueous medium. The negatively charged active agents are also referred to as “anionic active agents”. For instance, an active agent having a carboxylic acid group can typically take the form of —COOH in solid state and dissociates into a —COO⁻in an aqueous medium of appropriate pH. In other embodiments, the active agent may comprise charged functional groups such as —SO₃⁻, —PO₄²⁻, and the like.

Other active agents may be polarized or polarizable, that is, exhibiting a polarity at one portion relative to another portion.

The term “active agent” may also refer to electrically neutral agents, molecules, or compounds capable of being delivered via electro-osmotic flow. The electrically neutral agents are typically carried by the flow of a solvent. Selection of the suitable active agents is therefore within the knowledge of one skilled in the relevant art.

In some embodiments, one or more active agents may be selected from analgesics, anesthetics, anesthetics vaccines, antibiotics, adjuvants, immunological adjuvants, immunogens, tolerogens, allergens, toll-like receptor agonists, toll-like receptor antagonists, immuno-adjuvants, immuno-modulators, immuno-response agents, immuno-stimulators, specific immuno-stimulators, non-specific immuno-stimulators, and immuno-suppressants, or combinations thereof.

Non-limiting examples of such active agents include Lidocaine®, articaine, and others of the -caine class; morphine, hydromorphone, fentanyl, oxycodone, hydrocodone, buprenorphine, methadone, and similar opioid agonists; sumatriptan succinate, zolmitriptan, naratriptan HCl, rizatriptan benzoate, almotriptan malate, frovatriptan succinate and other 5-hydroxytryptamine1 receptor subtype agonists; resiquimod, imiquidmod, and similar TLR 7 and 8 agonists and antagonists; domperidone, granisetron hydrochloride, ondansetron and such anti-emetic drugs; zolpidem tartrate and similar sleep inducing agents; L-dopa and other anti-Parkinson's medications; aripiprazole, olanzapine, quetiapine, risperidone, clozapine, and ziprasidone, as well as other neuroleptica; diabetes drugs such as exenatide; as well as peptides and proteins for treatment of obesity and other maladies.

Further non-limiting examples of anesthetic active agents or pain killers include ambucaine, amethocaine, isobutyl p-aminobenzoate, amolanone, amoxecaine, amylocaine, aptocaine, azacaine, bencaine, benoxinate, benzocaine, N,N-dimethylalanylbenzocaine, N, N-dimethylglycylbenzocaine, glycylbenzocaine, beta-adrenoceptor antagonists betoxycaine, bumecaine, bupivicaine, levobupivicaine, butacaine, butamben, butanilicaine, butethamine, butoxycaine, metabutoxycaine, carbizocaine, carticaine, centbucridine, cepacaine, cetacaine, chloroprocaine, cocaethylene, cocaine, pseudococaine, cyclomethycaine, dibucaine, dimethisoquin, dimethocaine, diperodon, dyclonine, ecognine, ecogonidine, ethyl aminobenzoate, etidocaine, euprocin, fenalcomine, fomocaine, heptacaine, hexacaine, hexocaine, hexylcaine, ketocaine, leucinocaine, levoxadrol, lignocaine, lotucaine, marcaine, mepivacaine, metacaine, methyl chloride, myrtecaine, naepaine, octacaine, orthocaine, oxethazaine, parenthoxycaine, pentacaine, phenacine, phenol, piperocaine, piridocaine, polidocanol, polycaine, prilocaine, pramoxine, procaine (Novocaine®), hydroxyprocaine, propanocaine, proparacaine, propipocaine, propoxycaine, pyrrocaine, quatacaine, rhinocaine, risocaine, rodocaine, ropivacaine, salicyl alcohol, tetracaine, hydroxytetracaine, tolycaine, trapencaine, tricaine, trimecaine tropacocaine, zolamine, a pharmaceutically acceptable salt thereof, and mixtures thereof.

In certain embodiments, the active agent may be a low molecular weight molecule (MW<1000). In certain aspects, the molecule may be a polar polyelectrolyte. In certain other aspects, the molecule may be hydrophilic. In other aspects, the molecule may be lipophilic. In certain embodiments, the high molecular weight polyelectrolytic active agents may be proteins, polypeptides or nucleic acids.

As noted above, the device described herein is particularly suitable for delivery of low molecular weight active agents. These active agents are likely to be taken up by the body's immune system or organs too rapidly for them to be effective.

The active agents suitable for the iontophoresis device described herein can be selected in part based on their ability to be covalently conjugated to a nanoparticle via a linker, as defined herein. As used here, the terms “nanoparticle conjugate”, “active agent conjugate”, “nanoparticle-conjugated active agent” all refer to nanoparticles covalently coupled to one or more active agents.

Many active agents have carboxylate functionality. “Carboxylate” refers to carboxylic acid and its derivatives, such as carboxylate ester and amide. The coupling between a carboxylate group and a hydroxy functionality of a linker can be achieved by esterification and transesterification to form an ester linkage, such methods are well known in the art.

Typically, an active agent can be enzymatically cleaved from a nanoparticles at the ester linkage site where the active agent and the linker are coupled. The skin is rich with various types of esterases that are capable of detaching the active agent and allowing it to be released into the circulation in its free form. Significantly, depending on the linker structure, the kinetics of the enzymatic cleavage may be modulated. For instance, the rate of cleavage is likely to be reduced for a linker moiety having steric hindrance at the ester linkage site, because such a linker moiety forms a less than perfect fit at the enzyme's active site.

Accordingly, iontophoretic delivery of nanoparticles conjugated with an active agent can effect controlled release of such active agent. “Controlled release”, as used herein, means a method and composition for making an active agent available to the biological system of a host. Controlled-release includes the use of instantaneous release, delayed release, and sustained release. “Instantaneous release” refers to immediate release to the biosystem of the host. “Delayed release” means the active ingredient is not made available to the host until some time delay after administration. “Sustained Release” generally refers to release of active agents whereby the level of active agent available to the host is maintained at some level over a period of time. In particular, sustained release formulations are designed to release a drug slowly and the release itself is generally the rate determining step.

Advantageously, the nanoparticle conjugates of the present device are transported across a biological interface, such as skin. Because subcutaneously accumulated nanoparticles are far less likely to be taken up by the body's immune system or organs than systemically administered nanoparticles, an active agent depot may be formed in the biological tissue beyond the biological interface. The release of the active agent in its free form are determined by the rate of the enzymatic cleavage and may be modulated by judicious design of the linker structure. “Active agent depot” refers to an accumulation or concentrated population of active agents or their conjugates within a confined area in the biological tissue following iontophoresis. The active agents can diffuse from the depot into the circulation in a sustained manner.

The iontophoresis device described herein therefore provides the transport of nanoparticle conjugates across a permeable biological interface under the electro-repulsive and electro-osmotic forces generated by the iontophoresis device.

Thus, a further embodiment describes a method of for transdermal administration of an active agent by iontophoresis, the method comprising: positioning an active electrode assembly and a counter electrode assembly of an iontophoresis device on a biological interface of a subject, the active electrode assembly further including an active electrode element operable to provide an electrical potential; and an inner active agent reservoir comprising a plurality of nanoparticles, each nanoparticles being conjugated to one or more active agents via respective linkers; and applying a sufficient amount of current to administer a therapeutically effective amount of the active agents conjugated to the nanoparticles in the subject for a limited period of time.

In a further embodiment, the method comprises releasing the active agent from the nanoparticles following the transdermal administration. Depending on the type of linker and the triggering event that cleaves the point of conjugation between the linker and the active agent, the active agent can be released accordingly to different profiles. Typically, the release profile of the active agent may be a controlled release or sustained release. Targeted release is also possible. In a preferred embodiment, the release of the active agent is triggered by a targeted treatment site, for instance, a tumor site. For instance, certain enzymes may populate the tumor site but are scarce elsewhere. Linkers designed to be cleaved by these enzymes can cause the release of the active agents at the tumor site.

As used herein and in the claims, the term “membrane” means a layer, barrier or material, which may, or may not be permeable. Unless specified otherwise, membranes may take the form a solid, liquid or gel, and may or may not have a distinct lattice or cross-linked structure.

As used herein and in the claims, the term “ion selective membrane” means a membrane that is substantially selective to ions, passing certain ions while blocking passage of other ions. An ion selective membrane for example, may take the form of a charge selective membrane, or may take the form of a semi-permeable membrane.

As used herein and in the claims, the term “ion selective membrane” or “charge selective membrane” means a membrane that substantially passes and/or substantially blocks ions based primarily on the polarity or charge carried by the ion. Charge selective membranes are typically referred to as ion exchange membranes, and these terms are used interchangeably herein and in the claims. Charge selective or ion exchange membranes may take the form of a cation exchange membrane, an anion exchange membrane, and/or a bipolar membrane. A cation exchange membrane permits only the passage of cations and substantially blocks anions. Examples of commercially available cation exchange membranes include those available under the designators NEOSEPTA, CM-1, CM-2,CMX, CMS, and CMB from Tokuyama Co., Ltd. Conversely, An anion exchange membrane permits only the passage of anions and substantially blocks cations. Examples of commercially available anion exchange membranes include those available under the designators NEOSEPTA, AM-1, AM-3, AMX, AHA, ACH and ACS also from Tokuyama Co., Ltd.

As used herein and in the claims, term “bipolar membrane” means a membrane that is selective to two different charges or polarities. Unless specified otherwise, a bipolar membrane may take the form of a unitary membrane structure or multiple membrane structure. The unitary membrane structure may have a first portion including cation ion exchange material or groups and a second portion opposed to the first portion, including anion ion exchange material or groups. The multiple membrane structure (e.g., two film) may be formed by a cation exchange membrane attached or coupled to an anion exchange membrane. The cation and anion exchange membranes initially start as distinct structures, and may or may not retain their distinctiveness in the structure of the resulting bipolar membrane.

As used herein and in the claims, the term “semi-permeable membrane” means a membrane that substantially selective based on a size or molecular weight of the ion. Thus, a semi-permeable membrane substantially passes ions of a first molecular weight or size, while substantially blocking passage of ions of a second molecular weight or size, greater than the first molecular weight or size.

As used herein and in the claims, the term “porous membrane” means a membrane that is not substantially selective with respect to ions at issue. For example, a porous membrane is one that is not substantially selective based on polarity, and not substantially selective based on the molecular weight or size of a subject element or compound.

As used herein and in the claims, the term “reservoir” means any form of mechanism to retain an element or compound in a liquid state, solid state, gaseous state, mixed state and/or transitional state. For example, unless specified otherwise, a reservoir may include one or more cavities formed by a structure, and may include one or more ion exchange membranes, semi-permeable membranes, porous membranes and/or gels if such are capable of at least temporarily retaining an element or compound. Typically, a reservoir serves to retain a biologically active agent prior to the discharge of such agent by electromotive force into the biological interface. A reservoir may also retain an electrolyte solution.

The headings provided herein are for convenience only and do not interpret the scope or meaning of the embodiments.

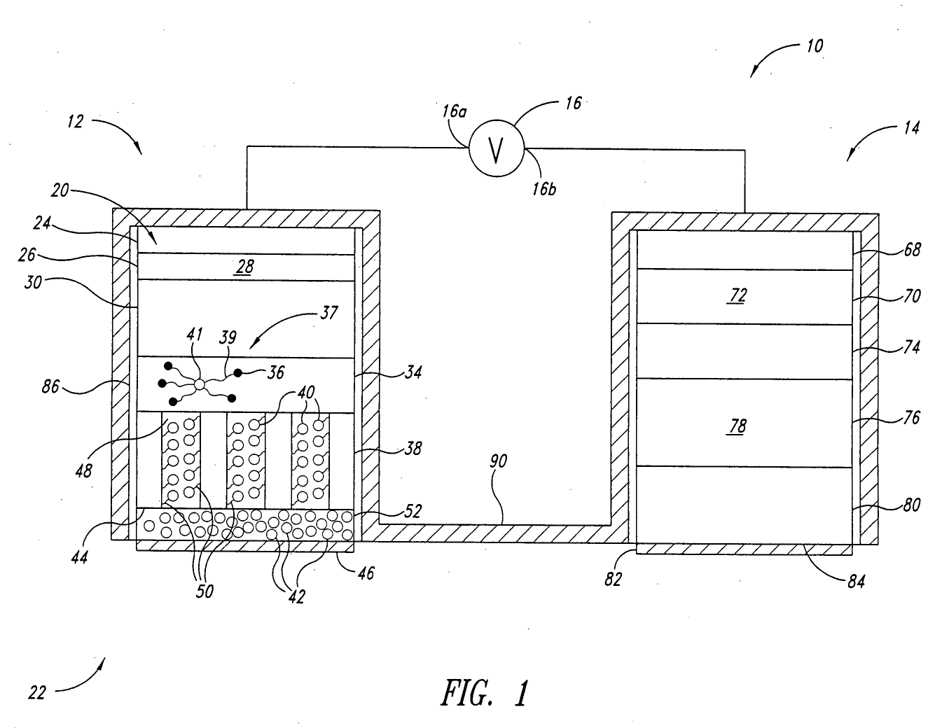

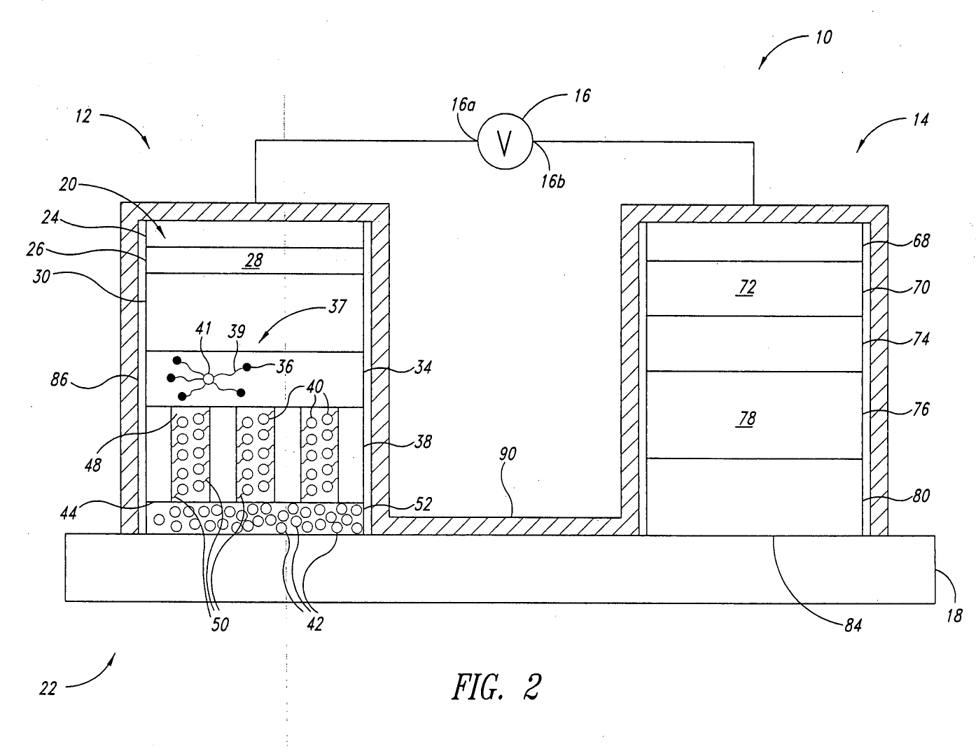

FIGS. 1 and 2 show an iontophoresis device 10 comprising active and counter electrode assemblies, 12, 14, respectively, electrically coupled to a power source 16, operable to supply an active agent contained in the active electrode assembly 12 to a biological interface 18 (FIG. 2), such as a portion of skin or mucous membrane via iontophoresis, according to one illustrated embodiment.

In the illustrated embodiment, the active electrode assembly 12 comprises, from an interior 20 to an exterior 22 of the active electrode assembly 12: an active electrode element 24, an optional electrolyte reservoir 26 storing an electrolyte 28, an optional inner ion selective membrane 30, an inner active agent reservoir 34 storing active agent 36 conjugated to a nanoparticle 41 via a linker 39, an optional outermost ion selective membrane 38 that optionally caches additional active agent 40, an optional further active agent 42 carried by an outer surface 44 of the outermost ion selective membrane 38, and an outer release liner 46. Unless specified otherwise, active agents 36, 40 and 42, when referred below, are considered to be in either conjugated or free form. Each of the above elements or structures will be discussed in detail below.

The active electrode element 24 is coupled to a first pole 16a of the voltage source 16 and positioned in the active electrode assembly 12 to apply an electromotive force or current to transport nanoparticle-conjugated active agent 36, 40, 42 via various other components of the active electrode assembly 12. The active electrode element 24 may take a variety of forms. For example, the active electrode element 24 may include a sacrificial element such as a chemical compound or amalgam including silver (Ag) or silver chloride (AgCl). Such compounds or amalgams typically employ one or more heavy metals, for example lead (Pb), which may present issues with regard manufacturing, storage, use and/or disposal. Consequently, some embodiments may advantageously employ a non-metallic active electrode element. Such may, for example, comprise multiple layers, for example a polymer matrix comprising carbon and a conductive sheet comprising carbon fiber or carbon fiber paper, such as that described in commonly assigned pending Japanese patent application 2004/317317, filed Oct. 29, 2004.

The inner active agent reservoir 34 is generally positioned between the inner ion selective membrane 30 and the outermost ion selective membrane 38, if such ion selective membranes are to be employed. The inner active agent reservoir 34 may take a variety of forms including any structure capable of temporarily retaining the nanoparticle conjugate 37. For example, the inner active agent reservoir 34 may take the form of a pouch or other receptacle, a membrane with pores, cavities or interstices, particularly where the nanoparticle conjugate 37 is in a liquid dispersion. The nanoparticle conjugate 37 comprises a nanoparticle 41 (e.g., gold) coupled to a number of active agents 36 via respective linkers 39. (Only one such conjugate is shown for sake of clarity of illustration.) The active electrode assembly 12 may optionally comprise an electrolyte reservoir 26 positioned between the active electrode element 24 and the outermost active electrode ion selective membrane 38, proximate the active electrode element 24. The electrolyte 28 contained therein may provide ions or donate charges to prevent or inhibit the formation of gas bubbles (e.g., hydrogen) on the active electrode element 24 in order to enhance efficiency and/or increase delivery rates. This elimination or reduction in electrolysis may in turn inhibit or reduce the formation of acids and/or bases (e.g., H⁺ions, OH⁻ions), that would otherwise present possible disadvantages such as reduced efficiency, reduced transfer rate, and/or possible irritation of the biological interface 18. As discussed in further details below, in some embodiments the electrolyte 28 may provide or donate ions to substitute for the active agent in the outermost ion selective membrane 38, for example substituting for the active agent 40 bonded to the ion exchange groups 50 where the outermost active electrode ion selective membrane 38 takes the form of an ion exchange membrane. Such may facilitate transfer of the active agent 40 to the biological interface 18, for example, increasing and/or stabilizing delivery rates. A suitable electrolyte may take the form of a solution of 0.5M disodium fumarate:0.5M poly(acrylic acid) (5:1).

Optionally, an inner ion selective membrane 30 can be positioned to separate the electrolyte 28 and the inner active agent reservoir 34. The inner ion selective membrane 30 may take the form of a charge selective membrane. For example, if the nanoparticles 41 are positively charged, the inner ion selective membrane 30 may take the form of an anion exchange membrane, selective to substantially pass anions and substantially block cations. The inner ion selective membrane 30 may advantageously prevent transfer of undesirable elements or compounds between the electrolyte 28 and the inner active agent reservoir 34. For example, the inner ion selective membrane 30 may prevent or inhibit the transfer of sodium (Na⁺), proton (H⁺) ions from the electrolyte 28, thereby increases the transfer rate and/or biological compatibility of the iontophoresis device 10.

Optionally, an outermost ion selective membrane 38 can be positioned generally opposed across the active electrode assembly 12 from the active electrode element 24. The outermost membrane 38 may, as in the embodiment illustrated in FIGS. 1 and 2, take the form of an ion exchange membrane, pores 48 (only one called out in FIGS. 1 and 2 for sake of clarity of illustration) of the ion selective membrane 38 including ion exchange material or groups 50 (only three called out in FIGS. 1 and 2 for sake of clarity of illustration). Under the influence of an electromotive force or current, the ion exchange material or groups 50 selectively substantially passes ions of the same polarity as conjugated active agent 36, 40, respectively, while substantially blocking ions of the opposite polarity. Thus, the outermost ion exchange membrane 38 is charge selective. Where nanoparticles 41 are cationic, the outermost ion selective membrane 38 may take the form of a cation exchange membrane. Alternatively, where the nanoparticles 41 are anionic, the outermost ion selective membrane 38 may take the form of an anion exchange membrane. As noted above, the nanoparticles described herein may also be neutral, in which case, the ion exchange membranes may nonetheless employed to block the undesirable ions from entering the inner active agent reservoir which may impede the transportation of the active agent.

The outermost ion selective membrane 38 may advantageously cache active agent 40, optionally conjugated to nanoparticles. In particular, the ion exchange groups or material 50 temporarily retains ions of the same polarity as the polarity of the active agent in the absence of electromotive force or current and substantially releases those ions when replaced with substitutive ions of like polarity or charge under the influence of an electromotive force or current.

The outermost ion selective membrane 38 may also be preloaded with the additional active agent 40, optionally conjugated to nanoparticles. Where the outermost ion selective membrane 38 is an ion exchange membrane, a substantial amount of active agent 40 may bond to ion exchange groups 50 in the pores, cavities or interstices 48 of the outermost ion selective membrane 38.

The active agent 42 that fails to bond to the ion exchange groups of material 50 may adhere to the outer surface 44 of the outermost ion selective membrane 38 as the further active agent 42. Alternatively, or additionally, the further active agent 42 may be positively deposited on and/or adhered to at least a portion of the outer surface 44 of the outermost ion selective membrane 38, for example, by spraying, flooding, coating, electrostatically, vapor deposition, and/or otherwise. In some embodiments, the further active agent 42 may sufficiently cover the outer surface 44 and/or be of sufficient thickness so as to form a distinct layer 52. In other embodiments, the further active agent 42 may not be sufficient in volume, thickness or coverage as to constitute a layer in a conventional sense of such term.

The active agent 42, whether in conjugated or in free form, may be deposited in a variety of highly concentrated forms such as, for example, solid form, nearly saturated solution form or gel form. If in solid form, a source of hydration may be provided, either integrated into the active electrode assembly 12, or applied from the exterior thereof just prior to use. If the active agent 42 is conjugated to nanoparticles, the solid form should be capable of forming a stable dispersion of individual nanoparticles upon hydration.

In some embodiments, the active agent 36, additional active agent 40, and/or further active agent 42 may be identical or similar compositions or elements. In other embodiments, the active agent 36, additional active agent 40, and/or further active agent 42 may be different compositions or elements from one another. In some embodiment, one or all of the active agents 36, 40 and 42 are conjugated to nanoparticles, with the proviso that at least one of them is conjugated to nanoparticles. Thus, a first type of active agent may be stored in the inner active agent reservoir 34, while a second type of active agent may be cached in the outermost ion selective membrane 38. In such an embodiment, either the first type or the second type of active agent may be deposited on the outer surface 44 of the outermost ion selective membrane 38 as the further active agent 42. Alternatively, a mix of the first and the second types of active agent may be deposited on the outer surface 44 of the outermost ion selective membrane 38 as the further active agent 42. As a further alternative, a third type of active agent composition or element may be deposited on the outer surface 44 of the outermost ion selective membrane 38 as the further active agent 42. In another embodiment, a first type of active agent may be stored in the inner active agent reservoir 34 as the active agent 36 and cached in the outermost ion selective membrane 38 as the additional active agent 40, while a second type of active agent may be deposited on the outer surface 44 of the outermost ion selective membrane 38 as the further active agent 42. Typically, in embodiments where one or more different active agents are employed, the respective nanoparticles conjugated to active agents 36, 40, 42 will all be of common polarity to prevent the active agents 36, 40, 42 from competing with one another. Other combinations are possible.

The outer release liner 46 may generally be positioned overlying or covering further active agent 42 carried by the outer surface 44 of the outermost ion selective membrane 38. The outer release liner 46 may protect the further active agent 42 and/or outermost ion selective membrane 38 during storage, prior to application of an electromotive force or current. The outer release liner 46 may be a selectively releasable liner made of waterproof material, such as release liners commonly associated with pressure sensitive adhesives. Note that the inner release liner 46 is shown in place in FIG. 1 and removed in FIG. 2.

An interface-coupling medium (not shown) may be employed between the electrode assembly and the biological interface 18. The interface-coupling medium may, for example, take the form of an adhesive and/or gel. The gel may, for example, take the form of a hydrating gel.

In the embodiment illustrated in FIGS. 1 and 2, the counter electrode assembly comprises, in order to form an interior 64 to an exterior 66 of the counter electrode assembly 14: a counter electrode element 68, electrolyte reservoir 70 storing an electrolyte 72, an inner ion selective membrane 74, an optional buffer reservoir 76 storing buffer material 78, an optional outermost ion selective membrane 80, and an optional outer release liner 82.

The counter electrode element 68 is electrically coupled to a second pole 16b of the voltage source 16, the second pole 16b having an opposite polarity to the first pole 16a. The counter electrode element 68 may take a variety of forms. For example, the counter electrode element 68 may include a sacrificial element, such as a chemical compound or amalgam including silver (Ag) or silver chloride (AgCl), or may include a non-sacrificial element such as the carbon-based electrode element discussed above.

The electrolyte reservoir 70 may take a variety of forms including any structure capable of retaining electrolyte 72, and in some embodiments may even be the electrolyte 72 itself, for example, where the electrolyte 72 is in a gel, semi-solid or solid form. For example, the electrolyte reservoir 70 may take the form of a pouch or other receptacle, or a membrane with pores, cavities or interstices, particularly where the electrolyte 72 is a liquid.

The electrolyte 72 is generally positioned between the counter electrode element 68 and the outermost ion selective membrane 80, proximate the counter electrode element 68. As described above, the electrolyte 72 may provide ions or donate charges to prevent or inhibit the formation of gas bubbles (e.g., hydrogen) on the counter electrode element 68 and may prevent or inhibit the formation of acids or bases or neutralize the same, which may enhance efficiency and/or reduce the potential for irritation of the biological interface 18.

The inner ion selective membrane 74 is positioned between and/or to separate, the electrolyte 72 from the buffer material 78. The inner ion selective membrane 74 may take the form of a charge selective membrane, such as the illustrated ion exchange membrane that substantially allows passage of ions of a first polarity or charge while substantially blocking passage of ions or charge of a second, opposite polarity. The inner ion selective membrane 74 will typically pass ions of opposite polarity or charge to those passed by the outermost ion selective membrane 80 while substantially blocking ions of like polarity or charge. Alternatively, the inner ion selective membrane 74 may take the form of a semi-permeable or microporous membrane that is selective based on size.

The inner ion selective membrane 74 may prevent transfer of undesirable elements or compounds into the buffer material 78. For example, the inner ion selective membrane 74 may prevent or inhibit the transfer of hydroxy (OH⁻) or chloride (Cl⁻) ions from the electrolyte 72 into the buffer material 78.

The optional buffer reservoir 76 is generally disposed between the electrolyte reservoir and the outermost ion selective membrane 80. The buffer reservoir 76 may take a variety of forms capable of temporarily retaining the buffer material 78. For example, the buffer reservoir 76 may take the form of a cavity, a porous membrane or a gel.

The buffer material 78 may supply ions for transfer through the outermost ion selective membrane 42 to the biological interface 18. Consequently, the buffer material 78 may, for example, comprise a salt (e.g., NaCl).

The outermost ion selective membrane 80 of the counter electrode assembly 14 may take a variety of forms. For example, the outermost ion selective membrane 80 may take the form of a charge selective ion exchange membrane, such as a cation exchange membrane or an anion exchange membrane, which substantially passes and/or blocks ions based on the charge carried by the ion. Examples of suitable ion exchange membranes are discussed above. Alternatively, the outermost ion selective membrane 80 may take the form of a semi-permeable membrane that substantially passes and/or blocks ions based on size or molecular weight of the ion.

The outermost ion selective membrane 80 of the counter electrode assembly 14 is selective to ions with a charge or polarity opposite to that of the outermost ion selective membrane 38 of the active electrode assembly 12. Thus, for example, where the outermost ion selective membrane 38 of the active electrode assembly 12 allows passage of negatively charged ions of the active agent 36, 40, 42 to the biological interface 18, the outermost ion selective membrane 80 of the counter electrode assembly 14 allows passage of positively charged ions to the biological interface 18, while substantially blocking passage of ions having a negative charge or polarity. On the other hand, where the outermost ion selective membrane 38 of the active electrode assembly 12 allows passage of positively charged ions of the active agent 36, 40, 42 to the biological interface 18, the outermost ion selective membrane 80 of the counter electrode assembly 14 allows passage of negatively charged ions to the biological interface 18 while substantially blocking passage of ions with a positive charge or polarity.

The outer release liner 82 may generally be positioned overlying or covering an outer surface 84 of the outermost ion selective membrane 80. Note that the inner release liner 82 is shown in place in FIG. 1 and removed in FIG. 2. The outer release liner 82 may protect the outermost ion selective membrane 80 during storage, prior to application of an electromotive force or current. The outer release liner 82 may be a selectively releasable liner made of waterproof material, such as release liners commonly associated with pressure sensitive adhesives. In some embodiments, the outer release liner 82 may be coextensive with the outer release liner 46 of the active electrode assembly 12.

The power source 16 may take the form of one or more chemical battery cells, super- or highly-capacitors, or fuel cells. The power source 16 may, for example, provide a voltage of 12.8V DC, with tolerance of 0.8V DC, and a current of 0.3 mA. The power source 16 may be selectively electrically coupled to the active and counter electrode assemblies 12a, 14 via a control circuit, for example, via carbon fiber ribbons. The iontophoresis device 10 may include discrete and/or integrated circuit elements to control the voltage, current and/or power delivered to the electrode assemblies 12, 14. For example, the iontophoresis device 10 may include a diode to provide a constant current to the electrode elements 20, 40.

As suggested above, the respective nanoparticles carriers of active agent 36, 40, 42 may be cationic or anionic. Consequently, the terminals or poles 16a, 16b of the voltage source 16 may be reversed. Likewise, the selectivity of the outermost ion selective membranes 38, 80 and inner ion selective membranes 30, 74 may be reversed.

The iontophoresis device 10 may further comprise an inert molding material 86 adjacent exposed sides of the various other structures forming the active and counter electrode assemblies 12, 14. The molding material 86 may advantageously provide environmental protection to the various structures of the active and counter electrode assemblies 12, 14.

As best seen in FIG. 2, the active and counter electrode assemblies 12,14 are positioned on the biological interface 18. Positioning on the biological interface may close the circuit, allowing electromotive force to be applied and/or current to flow from one pole 16a of the voltage source 16 to the other pole 16b, via the active electrode assembly, biological interface 18 and counter electrode assembly 14.

In the presence of the electromotive force and/or current, active agent 36 conjugated to nanoparticles (such as gold) are transported toward the biological interface 18. Additional active agent 40 is released by the ion exchange groups or material 50 by the substitution of ions of the same charge or polarity (e.g., active agent 36), and transported toward the biological interface 18. While some of the active agent 36 may substitute for the additional active agent 40, some of the active agent 36 may be transferred through the outermost ion elective membrane 38 into the biological interface 18. Further active agent 42 carried by the outer surface 44 of the outermost ion elective membrane 38 is also transferred to the biological interface 18.

The above description of illustrated embodiments, including what is described in the Abstract, is not intended to be exhaustive or to limit the claims to the precise forms disclosed. Although specific embodiments of and examples are described herein for illustrative purposes, various equivalent modifications can be made without departing from the spirit and scope of the invention, as will be recognized by those skilled in the relevant art. The teachings provided herein of the invention can be applied to other agent delivery systems and devices, not necessarily the exemplary iontophoresis active agent system and devices generally described above. For instance, some embodiments may omit some of the reservoirs, membranes or other structures. For example, some embodiment may include a control circuit or subsystem to control a voltage, current or power applied to the active and counter electrode elements 20, 40. Also for example, some embodiments may include an interface layer interposed between the outermost active electrode ion selective membrane 38 and the biological interface 18. Some embodiments may comprise additional ion selective membranes, ion exchange membranes, semi-permeable membranes and/or porous membranes, as well as additional reservoirs for electrolytes and/or buffers.

Various electrically conductive hydrogels have been known and used in the medical field to provide an electrical interface to the skin of a subject or within a device to couple electrical stimulus into the subject. Hydrogels hydrate the skin, thus protecting against burning due to electrical stimulation through the hydrogel, while swelling the skin and allowing more efficient transfer of an active component. Examples of such hydrogels are disclosed in U.S. Pat. Nos. 6,803,420; 6,576,712; 6,908,681; 6,596,401; 6,329,488; 6,197,324; 5,290,585; 6,797,276; 5,800,685; 5,660,178; 5,573,668; 5,536,768; 5,489,624; 5,362,420; 5,338,490; and 5,240995, herein incorporated in their entirety by reference. Further examples of such hydrogels are disclosed in U.S. Patent applications 2004/166147; 2004/105834; and 2004/247655, herein incorporated in their entirety by reference. Product brand names of various hydrogels and hydrogel sheets include Corplex™by Corium, Tegagel™ by 3M, PuraMatrix™ by BD; Vigilon™ by Bard; ClearSite™ by Conmed Corporation; FlexiGel™ by Smith & Nephew; Derma-Gel™ by Medline; Nu-Gel™ by Johnson & Johnson; and Curagel™ by Kendall, or acrylhydrogel films available from Sun Contact Lens Co., Ltd.

The various embodiments discussed above may advantageously employ various microstructures, for example microneedles. Microneedles and microneedle arrays, their manufacture, and use have been described. Microneedles, either individually or in arrays, may be hollow; solid and permeable; solid and semi-permeable; or solid and non-permeable. Solid, non-permeable microneedles may further comprise grooves along their outer surfaces. Microneedle arrays, comprising a plurality of microneedles, may be arranged in a variety of configurations, for example rectangular or circular. Microneedles and microneedle arrays may be manufactured from a variety of materials, including silicon; silicon dioxide; molded plastic materials, including biodegradable or non-biodegradable polymers; ceramics; and metals. Microneedles, either individually or in arrays, may be used to dispense or sample fluids through the hollow apertures, through the solid permeable or semi-permeable materials, or via the external grooves. Microneedle devices are used, for example, to deliver a variety of compounds and compositions to the living body via a biological interface, such as skin or mucous membrane. In certain embodiments, the active agent compounds and compositions may be delivered into or through the biological interface. For example, in delivering compounds or compositions via the skin, the length of the microneedle(s), either individually or in arrays, and/or the depth of insertion may be used to control whether administration of a compound or composition is only into the epidermis, through the epidermis to the dermis, or subcutaneous. In certain embodiments, microneedle devices may be useful for delivery of high-molecular weight active agents, such as those comprising proteins, peptides and/or nucleic acids, and corresponding compositions thereof. In certain embodiments, for example wherein the fluid is an ionic solution, microneedle(s) or microneedle array(s) can provide electrical continuity between a power source and the tip of the microneedle(s). Microneedle(s) or microneedle array(s) may be used advantageously to deliver or sample compounds or compositions by iontophoretic methods, as disclosed herein. In certain embodiments, for example, a plurality of microneedles in an array may advantageously be formed on an outermost biological interface-contacting surface of an iontophoresis device. Compounds or compositions delivered or sampled by such a device may comprise, for example, high-molecular weight active agents, such as proteins, peptides and/or nucleic acids.

In certain embodiments, compounds or compositions can be delivered by an iontophoresis device comprising an active electrode assembly and a counter electrode assembly, electrically coupled to a power source to deliver an active agent to, into, or through a biological interface. The active electrode assembly includes the following: a first electrode member connected to a positive electrode of the power source; an active agent reservoir having an active agent solution that is in contact with the first electrode member and to which is applied a voltage via the first electrode member; a biological interface contact member, which may be a microneedle array and is placed against the forward surface of the active agent reservoir; and a first cover or container that accommodates these members. The counter electrode assembly includes the following: a second electrode member connected to a negative electrode of the power source; an electrolyte reservoir that holds an electrolyte that is in contact with the second electrode member and to which voltage is applied via the second electrode member; and a second cover or container that accommodates these members.

In certain other embodiments, compounds or compositions can be delivered by an iontophoresis device comprising an active electrode assembly and a counter electrode assembly, electrically coupled to a power source to deliver an active agent to, into, or through a biological interface. The active electrode assembly includes the following: a first electrode member connected to a positive electrode of the power source; a first electrolyte reservoir having an electrolyte that is in contact with the first electrode member and to which is applied a voltage via the first electrode member; a first anion-exchange membrane that is placed on the forward surface of the first electrolyte reservoir; an active agent reservoir that is placed against the forward surface of the first anion-exchange membrane; a biological interface contacting member, which may be a microneedle array and is placed against the forward surface of the active agent reservoir; and a first cover or container that accommodates these members. The counter electrode assembly includes the following: a second electrode member connected to a negative electrode of the power source; a second electrolyte reservoir having an electrolyte that is in contact with the second electrode member and to which is applied a voltage via the second electrode member; a cation-exchange membrane that is placed on the forward surface of the second electrolyte reservoir; a third electrolyte reservoir that is placed against the forward surface of the cation-exchange membrane and holds an electrolyte to which a voltage is applied from the second electrode member via the second electrolyte reservoir and the cation-exchange membrane; a second anion-exchange membrane placed against the forward surface of the third electrolyte reservoir; and a second cover or container that accommodates these members.

Certain details of microneedle devices, their use and manufacture, are disclosed in U.S. Pat. Nos. 6,256,533; 6,312,612; 6,334,856; 6,379,324; 6,451,240; 6,471,903; 6,503,231; 6,511,463; 6,533,949; 6,565,532; 6,603,987; 6,611,707; 6,663,820; 6,767,341; 6,790,372; 6,815,360; 6,881,203; 6,908,453; 6,939,311; all of which are incorporated herein by reference in their entirety. Some or all of the teaching therein may be applied to microneedle devices, their manufacture, and their use in iontophoretic applications.

Aspects of the various embodiments can be modified, if necessary, to employ systems, circuits and concepts of the various patents, applications and publications to provide yet further embodiments, including those patents and applications identified herein. While some embodiments may include all of the membranes, reservoirs and other structures discussed above, other embodiments may omit some of the membranes, reservoirs or other structures. Still other embodiments may employ additional ones of the membranes, reservoirs and structures generally described above. Even further embodiments may omit some of the membranes, reservoirs and structures described above while employing additional ones of the membranes, reservoirs and structures generally described above.

The various embodiments described above can be combined to provide further embodiments. All of the U.S. patents, U.S. patent application publications, U.S. patent applications, foreign patents, foreign patent applications and non-patent publications referred to in this specification and/or listed in the Application Data Sheet are incorporated herein by reference, in their entirety, including but not limited to: Japanese patent application Serial No. H03-86002, filed Mar. 27,1991, having Japanese Publication No. H04-297277, issued on Mar. 3, 2000 as Japanese Patent No. 3040517; Japanese patent application Serial No.11-033076, filed Feb. 10, 1999, having Japanese Publication No. 2000-229128; Japanese patent application Serial No.11-033765, filed February 12,1999, having Japanese Publication No. 2000-229129; Japanese patent application Serial No. 11-041415, filed Feb. 19, 1999, having Japanese Publication No. 2000-237326; Japanese patent application Serial No. 11-041416, filed Feb. 19, 1999, having Japanese Publication No. 2000-237327; Japanese patent application Serial No. 11-042752, filed Feb. 22, 1999, having Japanese Publication No. 2000-237328; Japanese patent application Serial No. 11-042753, filed Feb. 22, 1999, having Japanese Publication No. 2000-237329; Japanese patent application Serial No. 11-099008, filed Apr. 6, 1999, having Japanese Publication No. 2000-288098; Japanese patent application Serial No. 11-099009, filed Apr. 6, 1999, having Japanese Publication No. 2000-288097; PCT patent application WO 2002JP4696, filed May 15, 2002, having PCT Publication No WO03037425; U.S. patent application Ser. No. 10/488970, filed Mar. 9, 2004; Japanese patent application 2004/317317, filed Oct. 29, 2004; U.S. provisional patent application Serial No. 60/627,952, filed Nov. 16, 2004; U.S. Provisional Patent Application No. 60/722,260, filed Sep. 30, 2005; Japanese patent application Serial No. 2004-347814, filed Nov. 30, 2004; Japanese patent application Serial No. 2004-357313, filed Dec. 9, 2004; Japanese patent application Serial No. 2005-027748, filed Feb. 3, 2005; and Japanese patent application Serial No. 2005-081220, filed Mar. 22, 2005.

Aspects of the various embodiments can be modified, if necessary, to employ systems, circuits and concepts of the various patents, applications and publications to provide yet further embodiments. While some embodiments may include all of the membranes, reservoirs and other structures discussed above, other embodiments may omit some of the membranes, reservoirs or other structures. Still other embodiments may employ additional ones of the membranes, reservoirs and structures generally described above. Even further embodiments may omit some of the membranes, reservoirs and structures described above while employing additional ones of the membranes, reservoirs and structures generally described above.

These and other changes can be made in light of the above-detailed description. In general, in the following claims, the terms used should not be construed to be limiting to the specific embodiments disclosed in the specification and the claims, but should be construed to include all systems, devices and/or methods that operate in accordance with the claims. Accordingly, the invention is not limited by the disclosure, but instead its scope is to be determined entirely by the following claims.

We/I claim:

1. An iontophoresis device for delivering active agents to a biological interface, the iontophoresis device comprising:
an active electrode assembly and a counter electrode assembly, the active electrode assembly including: an active electrode element operable to provide an electrical potential; and a first active agent reservoir comprising a plurality of nanoparticles, each nanoparticle being conjugated to one or more active agents via respective linkers.

2. The iontophoresis device of claim 1 wherein at least some of the nanoparticles are metallic.

3. The iontophoresis device of claim 2 wherein at least some of the nanoparticles are gold.

4. The iontophoresis device of claim 2 wherein at least some of the nanoparticles are silver or titanium oxide.

5. The iontophoresis device of claim 1 wherein at least some of the nanoparticles are solid, hollow shells or have core/shell structures.

6. The iontophoresis device of claim 1 wherein the nanoparticles have diameters of about 10-500 nm.

7. The iontophoresis device of claim 1 wherein at least some of the nanoparticles are coupled to the respective linkers by metal-sulfur bonds.

8. The iontophoresis device of claim 1 wherein at least some of the linkers are coupled to the respective active agents by carboxylate ester linkages.

9. The iontophoresis device of claim 1 wherein the linker is a poly(ethylene glycol) derivative.

10. The iontophoresis device of claim 9 wherein the poly(ethylene glycol) derivative has a molecular weight of about 500-2000 Daltons.

11. The iontophoresis device of claim 1 wherein at least some of the nanoparticles are charged.

12. The iontophoresis device of claim 1 wherein at least some of the nanoparticles are electrically neutral.

13. The iontophoresis device of claim 1, further comprising:
an electrolyte reservoir comprising an electrolyte composition; and
an inner ion selective membrane positioned between said electrolyte reservoir and said the first active agent reservoir.

14. The iontophoresis device of claim 13, further comprising:
an outermost ion selective membrane having an outer surface, the outer surface being proximate the biological interface when in use.

15. The iontophoresis device of claim 14, further comprising:
additional active agents cached in the outermost ion selective membrane.

16. The iontophoresis device of claim 15 wherein the additional active agents are conjugated to respective additional nanoparticles.

17. The iontophoresis device of claim 13, further comprising:
further active agents deposited on the outer surface of the outermost ion selective membrane.

18. The iontophoresis device of claim 17 wherein the further active agents are conjugated to respective further nanoparticles.

19. The iontophoresis device of claim 1 wherein the active agents can be released by enzymatic cleavage following the delivery.

20. The iontophoresis device of claim 1, further comprising:
one or more microneedles.

21. A method for transdermal administration of an active agent by iontophoresis, comprising:
positioning an active electrode assembly and a counter electrode assembly of an iontophoresis device on a biological interface of a subject, the active electrode assembly including: an active electrode element operable to provide an electrical potential; and a first active agent reservoir comprising a plurality of nanoparticles, each nanoparticle being conjugated to one or more active agents via respective linkers; and
applying a sufficient amount of current to administer a therapeutically effective amount of the active agents conjugated to the nanoparticles in the subject for a limited period of time.

22. The method of claim 21 wherein at least some of the nanoparticles are metallic.

23. The method of claim 21 wherein at least some of the nanoparticles are gold.

24. The method of claim 21 wherein the nanoparticles are coupled to the linkers by metal-sulfur bonds.

25. The method of claim 21 wherein the linkers are coupled to the active agents by carboxylate ester linkages.

26. The method of claim 21 wherein the linker is a poly(ethylene glycol) derivative.

27. The method of claim 21 wherein the nanoparticles remain conjugated to the respective active agents during the transdermal administration.
